(19) United States
(12) Patent Application Publication
(10) Pub. No.: US 2006/0172990 A1
(43) Pub. Date: Aug. 3, 2006

(54) Antibacterial agents

(76) Inventors: Stephen Peter East, Oxfordshire (GB); Ryan Ashley Bragg, Oxfordshire (GB); Steven Taylor, Oxfordshire (GB)

Correspondence Address: BANNER & WITCOFF, 1001 G STREET N W, SUITE 1100, WASHINGTON, DC, 20001, US

(21) Appl. No.: 10/538,928

(22) PCT Filed: Dec. 11, 2003
(86) PCT No.: PCT/GB03/05407

§ 371 Date: Mar. 3, 2006

(30) Foreign Application Priority Data
Dec. 19, 2002 (GB) 0229673.9

(51) Int. Cl. A61K 31/553; A61K 31/502

(52) U.S. Cl. 514/211.08; 514/575; 562/621; 514/357; 514/471; 514/406; 514/218; 514/247; 514/248; 540/545; 540/553; 544/237; 544/224

(57) ABSTRACT

Compounds of formula (I) have antibacterial activity, wherein Q represents —N(OH)CH(═O) or —C(═)NH(OH); Y represents —C(═O)—, —C(═S)—, —S(═O)—, or —SO₂—; R₁ represents hydrogen, C₁-C₆ alkyl or C1-C6 alkyl substituted by one or more halogen atoms, or, except when Q is a radical of formula —N(OH)CH(═O), a hydroxy, C₁-C₆ alkoxy, C₁-C₆ alkenyloxy, halogen, amino, C₁-C₆ alkylamino, or di-(C₁-C₆ alkyl)amino group; R₂ represents a substituted or unsubstituted C₁-C₆ alkyl, C₁-C₃ alkyl-O—C₁-C₃ alkyl, C₁-C₃ alkyl-C₁-C₃ alkyl, cycloalkyl(C₁-C₃ alkyl)-, aryl(C₁-C₃ alkyl)-, heterocyclyl(C₁-C₃ alkyl)-, or R¹R²N—C₁-C₃ alkyl group wherein R¹ represents hydrogen or C₁-C₃ alkyl and R² represents C₁-C₃ alkyl, or R¹R²N— represents a cyclic amino group; R₃ and R₅ independently represent hydrogen or a substituted or unsubstituted C₁-C₆ alkyl group or R₃ and R₅ taken together with the carbon and nitrogen atoms to which they are respectively attached form a saturated heterocyclic ring of from 5 to 7 ring atoms, which may be fused to a second carbocyclic or heterocyclic ring, either of which rings may optionally be substituted; R₄ represents hydrogen or a substituted or unsubstituted C₁-C₆ alkyl, C₂-C₆ alkenyl, C₂-C₆ alkynyl, cycloalkyl, aryl, heterocyclyl, C₁-C₃ alkyl-O—C₁-C₃ alkyl, C₁-C₃ alkyl-S—C₁-C₃ alkyl, C₁-C₃ alkyl-NH—(C₁-C₃ alkyl)-, cycloalkyl(C₁-C₃ alkyl)-, heterocyclic(C₁-C₃ alkyl)-group; and A represents a primary, secondary or tertiary amino group or a group —R₆, —OR₆, wherein R₆ is a substituted or unsubstituted C₁-C₆ alkyl, C₂-C₆ alkenyl, C₂-C₆ alkynyl, cycloalkyl, aryl, heterocyclyl, C₁-C₃ alkyl-O—(C₁-C₃ alky)-1, C₁-C₃ alkyl-S—(C₁-C₃ alkyl)-, C₁-C₃ alkyl-NH—(C₁-C₃ alkyl)-, cycloalkyl(C₁-C₃ alkyl)-, heterocyclic(C₁-C₃ alky)-1 or aryl(C₁-C₃ alkyl)-group.

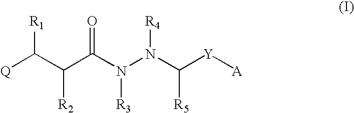

This invention relates to novel hydroxamic acid and N-formyl hydroxylamine derivatives having antibacterial activity, to methods of treatment using such compounds, and to pharmaceutical and veterinary compositions comprising such compounds.

### BACKGROUND TO THE INVENTION

Many classes of antibacterial agents are known, including the penicillins and cephalosporins, tetracyclines, sulfonamides, monobactams, fluoroquinolones and quinolones, aminoglycosides, glycopeptides, macrolides, polymyxins, lincosamides, trimethoprim and chloramphenicol. The fundamental mechanisms of action of these antibacterial classes vary.

Bacterial resistance to many known antibacterials is a growing problem. Accordingly there is a continuing need in the art for alternative antibacterial agents, especially those which have mechanisms of action fundamentally different from the known classes, and/or which are effective against the causative organisms of community acquired respiratory infections, and/or which are selective in their pharmacological activity, thus reducing risk of unwanted side effects.

Amongst the Gram-positive pathogens, such as staphylococci, streptococci, mycobacteria and enterococci, resistant strains have evolved/arisen which makes them particularly difficult to eradicate. Examples of such strains are methicillin resistant Staphylococcus aureus (MRSA), methicillin resistant coagulase negative Staphylococci (MRCNS), penicillin resistant Streptococcus pneumoniae and multiply resistant Enterococcus faecium

### BRIEF DESCRIPTION OF THE INVENTION

This invention makes available a new class of hydroxamic acid and N-formyl hydroxylamine derivatives having antibacterial activity. The compounds are characterised inter alia by the presence of a hydrazide feature in their structural backbone.

Although it may be of interest to establish the mechanism of action of the compounds with which the invention is concerned, it is their ability to inhibit bacterial growth that makes them useful. However, it is presently believed that their antibacterial activity is due, at least in part, to intracellular inhibition of bacterial polypeptide deformylase (PDF; EC 3.5.1.31).

### RELATED PRIOR ART

Although there are many publications disclosing both hydroxamic acid and N-formyl hydroxylamine derivatives as inhibitors of various metalloenzymes such as angiotensin converting enzyme, enkephalinase and the matrix metalloproteinases, there are relatively few relating to such compounds as antibacterial agents. The following patent publications are relevant in that connection:
WO 99/39704 (British Biotech)
WO 99/57097 (Versicor)
WO 99/59568 (British Biotech)
WO 00/35440 (British Biotech)
WO 00/44373 (British Biotech)
WO 00/58294 (British Biotech)
WO 00/61134 (British Biotech)
WO 01/10835 (British Biotech)
WO 01/38561 (Questcor)
WO 01/40198 (Aventis)
WO 01/42431 (Bayer)
WO 01/44178 (Versicor)
WO 01/44179 (Versicor)
WO 01/85160 (SmithKline Beecham)
WO 01/85170 (SmithKline Beecham)
WO 02/28829 (Questcor)
WO 02/41886 (British Biotech)
WO 02/50081 (British Biotech)
WO 02/070541 SmithKline Beecham) relates to PDF inhibitor compounds which have a hydrazide feature in the backbone and an N-formylhydroxylamino metal binding group.

In addition the natural antibiotic actinonin (see for example J.C.S Perkin 1,1975, 819) is a hydroxamic acid derivative of Structure (A): 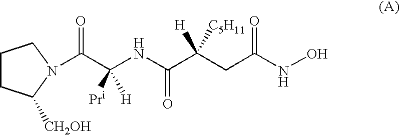
which is now known to act by inhibition of PDF. In addition to actinonin, various structural analogues of actinonin have also been shown to have antibacterial activity (see for example Deviun et al. Journal of the Chemical Society. Perkin Transactions 1 (9):830-841, 1975; Broughton et al. Journal of the Chemical Society. Perkin Transactions 1 (9):857-860, 1975).

The matlystatin group of compounds share a number of structural similarities with actinonin. Both are peptidic molecules with functional hydroxamic acid metal binding groups (Ogita et al., J. Antibiotics. 45(11):1723-1732; Tanzawa et al., J. Antibiotics. 45(11):1733-1737; Haruyama et al., J. Antibiotics. 47(12):1473-1480; Tamaki et al., J. Antibiotics. 47(12):1481-1492). The matlystatins and their close structural analogues are characterised by the presence in the molecule of a divalent piperazin-1,6-diyl group, i.e.

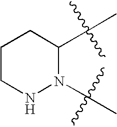

In view of their close structural similarity to actinonin, the observation that actinonin inhibits PDF implies that matlystatin compounds may also inhibit PDF.

For a recent review of peptide deformylase inhibitors, see Clements et. al., Curr. Med. Chem.—Anti-Infective Agents, 2002,1, 239-249.

The following patent publications disclose N-formyl hydroxylamine structures:
EP-B-0236872 (Roche)
WO 92/09563 (Glycomed)
WO 92/04735 (Syntex)
WO 95/19965 (Glycomed)
WO 95/22966 (Sanofi Winthrop)
WO 95/33709 (Roche)
WO 96/23791 (Syntex)
WO 96/16027 (Syntex/Agouron)
WO 97/03783 (British Biotech)
WO 97/18207 (DuPont Merck)
WO 98/38179 (GlaxoWellcome)
WO 98/47863 (Labs Jaques Logeais)

The pharmaceutical utility ascribed to the N-formyl hydroxylamine derivatives in those publications is the ability to inhibit matrix metalloproteinases (MMPs) and in some cases release of tumour necrosis factor (TNF), and hence the treatment of diseases or conditions mediated by those enzymes, such as cancer and rheumatoid arthritis.

In addition to these, U.S. Pat. No. 4,738,803 (Roques et al.) also discloses N-formyl hydroxylamine derivatives. However, these compounds are disclosed as enkephalinase inhibitors and are proposed for use as antidepressants and hypotensive agents. Also, WO 97/38705 (Bristol-Myers Squibb) discloses certain N-formyl hydroxylamine derivatives as enkephalinase and angiotensin converting enzyme inhibitors.

There are too many publications relating to metalloenzyme inhibiting hydroxylamic acid derivatives to summarise effectively. However a recent review, Whittaker et. al. Chem. Rev. 1999, 99, 2735, provides an overview of that art.

### DESCRIPTION OF THE INVENTION

The present invention provides a compound of formula (I) or a pharmaceutically or veterinarily acceptable salt, hydrate or solvate thereof 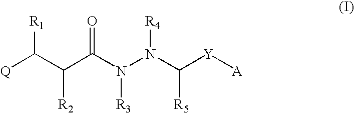
wherein

Q represents a radical of formula —N(OH)CH(═O) or formula —C(═O)NH(OH);

Y represents —C(═O)—, —C(═S), —S(═O)—, or —SO₂—;

R₁ represents hydrogen, C₁-C₆ alkyl or C₁-C₆ alkyl substituted by one or more halogen atoms, or, except when Q is a radical of formula —N(OH)CH(═O), a hydroxy, C₁-C₆ alkoxy, C₁-C₆ alkenyloxy, halogen, amino, C₁-C₆ alkylamino, or di-(C₁-C₆ alkyl)amino group

R₂ represents a substituted or unsubstituted C₁-C₆ alkyl, C₁-C₃ alkyl-O—C₁-C₃ alkyl, C₁-C₃ alkyl-S—C₁-C₃ alkyl, cycloalkyl(C₁-C₃ alkyly, aryl(C₁-C₃ alkyly, heterocyclyl(C₁-C₃ alkyl)-, or R¹R²N—C₁-C₃ alkyl group wherein R¹ represents hydrogen or C₁-C₃ alkyl and R² represents C₁-C₃ alkyl, or R¹R²N— represents a cyclic amino group;

R₃ and R₅ independently represent hydrogen or a substituted or unsubstituted C₁-C₆ alkyl group or R₃ and R₅ taken together with the carbon and nitrogen atoms to which they are respectively attached form a saturated heterocyclic ring of from 5 to 7 ring atoms, which may be fused to a second carbocyclic or heterocyclic ring, either of which rings may optionally be substituted;

R₄ represents hydrogen or a substituted or unsubstituted C₁-C₆ alkyl, C₂-C₆ alkenyl, C₂-C₆ alkynyl, cycloalkyl, aryl, heterocyclyl, C₁-C₃ alkyl-O—C₁-C₃ alkyl, C₁-C₃ alkyl-S—(C₁-C₃ alkyl)-, C₁-C₃ alkyl-NH—(C₁-C₃ alkyl)-, cycloalkyl(C₁-C₃ alkyl), heterocyclic(C₁-C₃ alkyl) or aryl(C₁-C₃ alkyl)-group; and

A represents a primary, secondary or tertiary amino group or a group —R₆, —OR₆, wherein R₆ is a substituted or unsubstituted C₁-C₆ alkyl, C₂-C6 alkenyl, C₂-C₆ alkynyl, cycloalkyl, aryl, heterocyclyl, C₁-C₃ alkyl-O—(C₁-C₃ alkyl), C₁-C₃ alkyl-S—(C₁-C₃ alkyl), C₁-C₃ alkyl-NH—(C₁-C₃ alky)-l, cycloalkyl(C₁-C₃ alkyl)-, heterocyclic(C₁-C₃ alkyl) or aryl(C₁-C₃ alkyl)-group.

In another aspect, the invention provides a method for the treatment of-bacterial infections in humans and non-human mammals, which comprises administering to a subject suffering such infection an antibacterially effective dose of a compound of formula (I) as defined above. Also included in the invention is the use of a compound of formula (I) as defined above for inhibiting bacterial growth in vitro and in vivo in mammals, and the use of such a compound for the manufacture of a composition for treating bacterial infection by inhibiting bacterial growth.

In a further aspect of the invention there is provided a method for the treatment of bacterial contamination by applying an antibacterially effective amount of a compound of formula (I) as defined above to the site of contamination.

The compounds of formula (I) as defined above may be used as component(s) of antibacterial cleaning or disinfecting materials.

As used herein terms of the form “(Cₐ-C_{b})alkyl” where a and b are integers refer to a straight or branched chain alkyl moiety having from a to b carbon atoms. Thus, for example, the term “(C₁-C₆)alkyl” means a straight or branched chain alkyl moiety having from 1 to 6 carbon atoms, and includes, for example, methyl, ethyl, n-propyl, isopropyl, n-butyl, isobutyl, sec-butyl, t-butyl, n-pentyl and n-hexyl.

As used herein terms of the form “divalent (Cₐ-C_{b})alkylene radical” where a and b are integers refer to a saturated hydrocarbon chain having from a to b carbon atoms and two unsatisfied valencies.

As used herein terms of the form “(Cₐ-C_{b})alkenyl” where a and b are integers refer to straight or branched chain alkenyl moiety having from a to b carbon atoms having at least one double bond of either E or Z stereochemistry where applicable. Thus, for example, the term “(C₁-C₆)alkenyl” means a straight or chain alkenyl moiety having from 2 to 6 carbon atoms having at least one double bond, and includes, for example, vinyl, allyl, 1- and 2-butenyl and 2-methyl-2-propenyl.

As used herein the term “Cₐ-C_{b} alkynyl” where a and b are integers refers to straight chain or branched chain hydrocarbon groups having from two to six carbon atoms and having in addition one triple bond. Thus, for example, the term “(C₁-C₆)alkynyl” would include for example, ethynyl, 1-propynyl, 1- and 2-butynyl, 2-methyl-2-propynyl, 2-pentynyl, 3-pentynyl, 4-pentynyl, 2-hexynyl, 3-hexynyl, 4-hexynyl and 5-hexynyl.

As used herein the term ”cycloalkyl” means a saturated alicyclic moiety having from 3-8 carbon atoms and includes, for example, cyclopropyl, cyclobutyl, cyclopentyl, cyclohexyl, cycloheptyl and cyclooctyl.

As used herein the term “aryl” refers to a mono-, bi- or tri-cyclic carbocyclic aromatic group, and to groups consisting of two covalently linked monocyclic carbocyclic aromatic groups. Illustrative of such groups are phenyl, biphenyl and napthyl.

As used herein the term “heteroaryl” refers to a 5- or 6-membered aromatic ring containing one or more heteroatoms. Illustrative of such groups are thienyl, furyl, pyrrolyl, imidazolyl, benzimidazolyl, thiazolyl, pyrazolyl, isoxazolyl, isothiazolyl, triazolyl, thiadiazolyl, oxadiazolyl, pyridinyl, pyridazinyl, pyrimidinyl, pyrazinyl, triazinyl.

As used herein the unqualified term “heterocyclyl” or “heterocyclic” includes “heteroaryl” as defined above, and in particular means a 5-7 membered aromatic or non-aromatic heterocyclic ring containing one or more heteroatoms selected from S, N and O, including for example, pyrrolyl, furanyl, thienyl, piperidinyl, imidazolyl, oxazolyl, isoxazolyl, thiazolyl, thiadiazolyl, pyrazolyl, pyridinyl, pyrrolidinyl, pyrimidinyl, morpholinyl, piperazinyl, indolyl, morpholinyl, benzofuranyl, pyranyl, isoxazolyl, benzimidazolyl, methylenedioxyphenyl, maleimido and succinimido groups.

Unless otherwise specified in the context in which it occurs, the term “substituted” as applied to any moiety herein means substituted with up to four substituents, each of which independently may be (C₁-C₆)alkyl, (C₁-C₆)alkoxy, hydroxy, mercapto, (C₁-C₆)alkylthio, amino, halo (including fluoro, chloro, bromo and iodo), trifluoromethyl, nitro, —COOH, —CONH₂, —COR^{A}, —COOR^{A}, —NHCOR^{A}, —CONHR^{A}, —NHR^{A}, —NR^{A}R^{B}, or —CONR^{A}R^{B} wherein R^{A} and R^{B} are independently a (C₁-C₆)alkyl group.

Salts of the compounds of the invention include physiologically acceptable acid addition salts for example hydrochlorides, hydrobromides, sulphates, methane sulphonates, p-toluenesulphonates, phosphates, acetates, citrates, succinates, lactates, tartrates, fumarates and maleates. Salts may also be formed with bases, for example sodium, potassium, magnesium, and calcium salts.

There are several actual or potential chiral centres in the compounds according to the invention because of the presence of asymmetric carbon atoms. The presence of several asymmetric carbon atoms gives rise to a number of diastereoisomers with R or S stereochemistry at each chiral centre. The invention includes all such diastereoisomers and mixtures thereof. Currently, the preferred stereoconfiguration of the carbon atom carrying the R₂ group is R.

In the compounds of the invention, in relation to the groups Q, R₁, R₂, R₃, R₄, Y and A, separately and in combination:

The group Q

It is currently preferred that Q is an N-formyl hydroxylamine group —N(OH)CH(═O).

The radical —Y—

It is currently preferred that —Y— is —C(═O)— or —SO₂—.

The group R₁

R₁ may be, for example, hydrogen, methyl, trifuoromethyl or, in the case where Q is a hydroxamic acid group HONHCO—, fluorine. Hydrogen is currently preferred.

The group R₂

R₂ may be, for example:
optionally substituted C₁-C₆ alkyl, C₃-C₆ alkenyl, C₃-C₆ alkynyl or cycloalkyl;
phenyl(C₁-C₆ alkyl), phenyl(C₃-C₆ alkenyl)- or phenyl(C₃-C₆ alkynyl)- optionally substituted in the phenyl ring;
cycloalkyl(C₁-C₆ alkyl)-, cycloalkyl(C₃-C₆ alkenyl)- or cycloalkyl(C₃-C6 alkynyl)- optionally substituted in the cycloalkyl ring; or
CH₃(CH₂)ₚO(CH₂)_{q}— or CH₃(CH₂)ₚS(CH₂)_{q}—, wherein p is 0, 1, 2 or 3 and q is 1, 2 or 3.

Specific examples of R₂ groups include
methyl, ethyl, n- and iso-propyl, n- and iso-butyl, n-pentyl, iso-pentyl, 3-methyl-but-1-yl, n-hexyl, n-heptyl, n-acetyl, n-octyl, methylsulfanylethyl, ethylsulfanylmethyl, 2-methoxyethyl, 2-ethoxyethyl, 2-ethoxymethyl, 3-hydroxypropyl, allyl, 3-phenylprop-3-en-1-yl, prop-2-yn-1-yl, 3-phenylprop-2-yn-1-yl, 3-(2-chlorophenyl)prop-2-yn-1-yl, but-2-yn-1-yl, cyclopentyl, cyclohexyl, cyclopentylmethyl, cyclopentylethyl, cyclopentylpropyl, cyclohexylmethyl, cyclohexylethyl, cyclohexylpropyl, furan-2-ylmethyl, furan-3-methyl, tetrahydrofuran-2-ylmethyl, tetrahydrofuran-2-ylmethyl, piperidinylmethyl, pyrid-2-ylmethyl, pyrid-3-ylmethyl, pyrid-4-ylmethyl, phenylpropyl, 4-chlorophenylpropyl, 4-methylphenylpropyl, 4-methoxyphenylpropyl, benzyl, 4-chlorobenzyl, 4-methylbenzyl, and 4-methoxybenzyl.
Presently preferred groups at R₂ are (C₁-C₆)alkyl-, cycloalkylmethyl-, (C₁-C₃)alkyl-S—(C₁-C₃)alkyl-, or (C₁-C₃)alkyl-O—(C₁-C₃)alkyl-, especially n-propyl, n-butyl, n-pentyl, cyclopentylmethyl, cyclopentylethyl, cyclohexylmethyl or cyclohexylethyl.

The Group R₄

Examples of the group R₄ include hydrogen, (C₁-C₆)alkyl-, cycloalkylmethyl-, (C₁-C₃)alkyl-S—(C₁-C₃)alkyl-, or (C₁-C₃)alkyl-O—(C₁-C₃)alkyl-, especially methyl, ethyl, n-propyl, n-butyl, n-pentyl, cyclopentylmethyl, cyclopentylethyl, cyclohexylmethyl or cyclohexylethyl.

R₃ and R₅ When Not Part of a Ring

When not part of a ring, R₃ and R₅ may independently be, for example, hydrogen, (C₁-C₆)alkyl-, cycloalkylmethyl-, (C₁-C₃)alkyl-S—(C₁-C₃)alkyl-, or (C_{1—-C}₃)alkyl-O—(C₁-C₃)alkyl-, especially methyl, ethyl, n-propyl, n-butyl, n-pentyl, cyclopentylmethyl, cyclopentylethyl, cyclohexylmethyl or cyclohexylethyl.

The Ring Formed by R₃ and R₅ and the Nitrogens to Which They Are Attached

Examples of rings which may be formed by R₃ and R₅ taken together with the carbon and nitrogen atoms to which they are respectively attached are the following, wherein R₄ is as defined in relation to formula (I) and R represents hydrogen or C₁-C₄ alkyl:

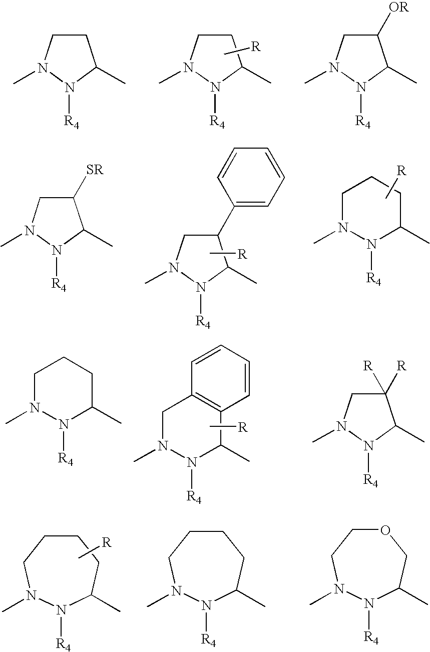

In the above rings, where a sulfur atom is present as a ring member, the equivalent structures wherein that sulfur is oxidised to —SO— or —SO₂— are also examples of ring structures which may be formed by R₃ and R₅.

The Group A

The group A is a primary, secondary or tertiary amino group or a group —R₆, or —OR₆. When A is —R₆, or —OR₆, the R₆ group may be, for example, any of those given as R₂ examples above, or a group of formula (II) as defined below, including such specific examples of groups (II) as morpholinyl, furanyl, thienyl, phenyl, and benzyl.

Presently it is preferred that A is a secondary or tertiary amino group, and in the latter case it may be a non-cyclic or a cyclic amino group. For example, A may be an amino group of formula —NR₆R₇ wherein R₆ and R₇ independently represent a radical of formula (IV) 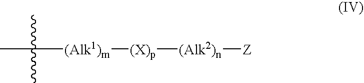
wherein

m, p and n are independently 0 or 1;

Z represents hydrogen or a carbocyclic or heterocyclic ring of 5 to 7 ring atoms which is optionally fused to a saturated or unsaturated carbocyclic or heterocyclic second ring of 5 to 7 ring atoms;

Alk¹ and Alk² independently represent divalent C₁-C₃ alkylene radicals;

X represents —O—, —S—, —S(O)—, —S(O₂)—, —C(═O)—, —NH—, —NR₇— where R₇ is C₁-C₃ alkyl;

and wherein

Alk¹, Alk² and Z when other than hydrogen, independently are optionally substituted by
(C₁-C₃)alkyl, (C₂-C₃)alkenyl, or (C₂-C₃)alkynyl, phenyl, optionally substituted by (C₁-C₃)alkyl, (C₁-C₃)alkoxy, halo, nitro, amino, mono- or di-(C₁-C₃)alkylamino, cyano or trifluoromethyl;
monocyclic 5 or 6-membered heterocyclic, optionally substituted by (C₁-C₃)alkyl, (C₁-C₃)alkoxy, halo, nitro, amino, mono- or di-(C₁-C₃)alkylamino, cyano or trifluoromethyl
benzyl, optionally substituted in the phenyl ring by (C₁-C₃)alkyl, (C₁-C₃)alkoxy, halo, nitro, amino, mono- or di-(C₁-C₃)alkylamino, cyano or trifluoromethyl,
hydroxy, phenoxy, (C₁-C₆)alkoxy, or hydroxy(C₁-C₆)alkyl,
mercapto, (C₁-C₆)alkylthio or mercapto(C₁-C₆)alkyl,
oxo,
nitro,
cyano
halo
—COOH, or —COOR^{A},
—CONH₂, —CONHR^{A}, or —CONR^{A}R^{B}
COR^{A}, —SO₂R^{A},
—NHCOR^{A},
—NH₂, —NHR^{A}, or —NR^{A}R^{B},
   wherein R^{A} and R^{B} are independently a (C₁-C₆) alkyl group, R^{A} and R^{B} taken together with the nitrogen atom to which they are attached form a 5- or 6-membered heterocyclic ring which may be substituted by (C₁-C₃)alkyl, hydroxy, or hydroxy(C₁-C₃)alkyl.

The amino group A may also be one of formula —NR₈R₉ wherein R₈ and R₉ when taken together with the nitrogen atom to which they are attached form a saturated heterocyclic ring of 5 to 8 atoms optionally fused to a saturated or unsaturated carbocyclic or heterocyclic second ring of 5 to 7 ring atoms, any of which rings being optionally substituted by a radical of formula (IV) as defined above. Examples of cyclic amino groups are 1-pyrrolidinyl, piperidin-1-yl, 1-piperazinyl, hexahydro-1-pyridazinyl, morpholin-4-yl, tetrahydro-1,4-thiazin-4-yl, tetrahydro-1,4-thiazin-4-yl 1-oxide, tetrahydro-1,4-thiazin-4-yl 1,1-dioxide, hexahydroazipino, thiomorpholino, diazepino, and thiazolidinyl. Presently preferred are piperidin-1-yl and 1-piperazinyl.

The Group of Formula (IV)

In the substituent (IV) Alk¹ and Alk2 may independently represent, for example —(CH₂)— or —(CH₂CH₂)—. In the case where m is 0 and p is 1, X may be, for example —S—, —S(═O)—, or preferably —C(═O)— or —S(O₂)—. In such cases n may be 0 or 1

Compounds of the invention include those selected from the group consisting of compounds of formulae (IIA)-(IID). 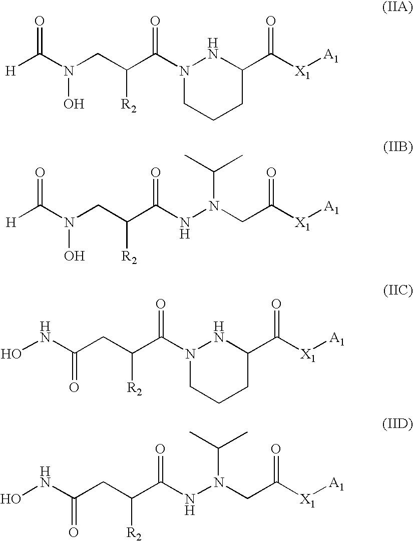
wherein R₂ is as defined in relation to formula (IV), especially n-propyl, n-butyl, n-pentyl, cyclopentylmethyl, cyclopentylethyl, cyclohexylmethyl or cyclohexylethyl;

X₁ is a bond, C₁-C₃ alkylene, —NH— or —O—; and

A₁ is optionally substituted C₁-C₆ alkyl, cycloalkyl, aryl, or heterocyclic, for example methyl, ethyl phenyl, cyclopentyl, cyclohexyl, 2- or 3-furanyl, 2- or 3-thienyl, 2-, 3- or 4-pyridyl, 3-, 4- or 5-pyrazolyl, 3-, 4- or 5-oxazolyl, or 3-, 4- or 5-thiazolyl, methoxymethyl, 3,5-bis-(trifluoromethyl)phenyl, 4-trifluoromethylphenyl, 4-methoxyphenyl, 3,4-methylenedioxyphenyl, 4-fluorophenyl benzyl, 3-pyridyl, 4-pyridyl, cyclohexyl, 1,3-dimethylpyrazol-5-yl, 1-methylimidazol-5-yl, and 2-[morpholin-1 -yl]pyrid-5-yl.

Particular compounds of the invention include those of the Examples herein.

In general, compounds of the invention are accessible by conventional synthetic procedures. For example, they may be prepared by condensation of an acid of formula (IIIA) with a hydrazide of formula (IIIB):

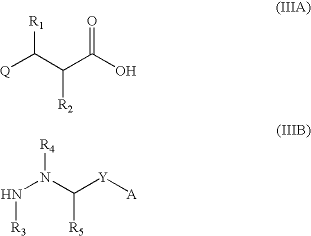

In addition, compounds wherein A is a primary, secondary or tertiary amino group may be prepared by condensation of a compound of formula (III) or an activated derivative thereof with the appropriate amine:

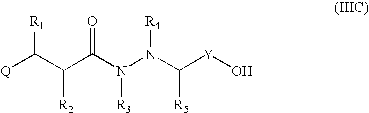

As is conventional, any reactive constituents of the intermediates used in the above reactions may be protected during the condensation reaction and deprotected subsequently.

The Examples herein provide further details of routes and methods for the preparation of compounds of the invention.

Compositions with which the invention is concerned may be prepared for administration by any route consistent with the pharmacokinetic properties of the active ingredient(s).

Orally administrable compositions may be in the form of tablets, capsules, powders, granules, lozenges, liquid or gel preparations, such as oral, topical, or sterile parenteral solutions or suspensions. Tablets and capsules for oral administration may be in unit dose presentation form, and may contain conventional excipients such as binding agents, for example syrup, acacia, gelatin, sorbitol, tragacanth, or polyvinyl-pyrrolidone; fillers for example lactose, sugar, maize-starch, calcium phosphate, sorbitol or glycine; tabletting lubricant, for example magnesium stearate, talc, polyethylene glycol or silica; disintegrants for example potato starch, or acceptable wetting agents such as sodium lauryl sulphate. The tablets may be coated according to methods well known in normal pharmaceutical practice. Oral liquid preparations may be in the form of, for example, aqueous or oily suspensions, solutions, emulsions, syrups or elixirs, or may be presented as a dry product for reconstitution with water or other suitable vehicle before use. Such liquid preparations may contain conventional additives such as suspending agents, for example sorbitol, syrup, methyl cellulose, glucose syrup, gelatin hydrogenated edible fats; emulsifying agents, for example lecithin, sorbitan monooleate, or acacia; non-aqueous vehicles (which may include edible oils), for example almond oil, fractionated coconut oil, oily esters such as glycerine, propylene glycol, or ethyl alcohol; preservatives, for example methyl or propyl p-hydroxybenzoate or sorbic acid, and if desired conventional flavouring or colouring agents.

Safe and effective dosages for different classes of patient and for different disease states will be determined by clinical trial as is required in the art. It will be understood that the specific dose level for any particular patient will depend upon a variety of factors including the activity of the specific compound employed, the age, body weight, general health, sex, diet, time of administration, route of administration, rate of excretion, drug combination and the severity of the particular disease undergoing therapy.

The following examples illustrate embodiments of the invention.

In the Examples, the following abbreviations have been used:
DMF Dimethylformamide
HOBt 1-Hydroxybenzotriazole
HPLC High performance liquid chromatography
LRMS Low resolution mass spectrometry
NMR Nuclear magnetic resonance
RT Retention Time
TLC Thin layer chromatography
DIEA N,N-diisopropylethylamine
DCM Dichloromethane
HATU O-(7-Azabenzotriazo-1-yl)-N,N,N′,N′-tetramethyluronium hexafluorophosphate

¹H and ¹³C NMR spectra were recorded using a Bruker DPX 250 spectrometer at 250.1 and 62.9 MHz, respectively. Mass spectra were obtained using a Perkin Elmer Sciex API 165 spectrometer using both positive and negative ionisation modes. Infra-red spectra were recorded on a Perkin Elmer PE 1600 FTIR spectrometer. Analytical HPLC was performed on a Beckman System Gold, using Waters Nova Pak C18 column (150 mm, 3.9 mm) with 20 to 90% solvent B gradient (1 ml/min) as the mobile phase. [Solvent A: 0.05% TFA in 10% water 90% methanol; Solvent B: 0.05% TFA in 10% methanol 90%], detection wavelength at 230 nm. Preparative HPLC was performed on a Gilson autoprep instrument using a C18 Waters delta prep-pak cartridge (15 μm, 300 A, 25 mm, 10 mm) with 20 to 90% solvent B gradient (6 ml/min) as the mobile phase. [Solvent A water; Solvent B: methanol], UV detection was at 230 nm.

### EXAMPLE 1

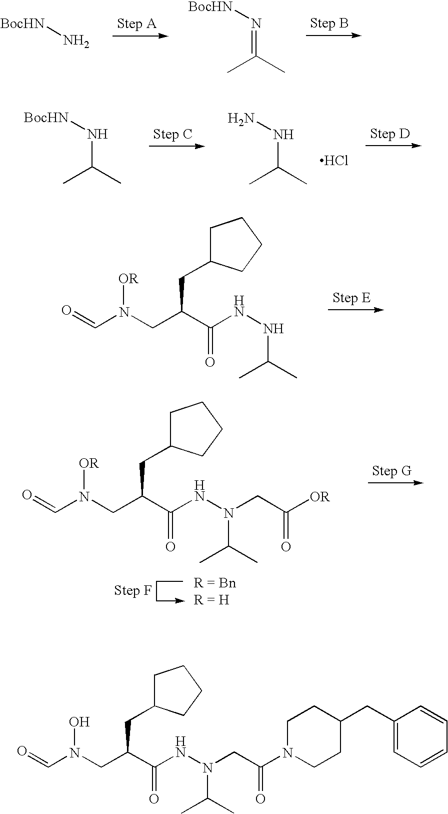

Step A: acetone, NaBH(OAc)₃, THF, rt, 16 h; Step B: H₂, 5% Pt/C, MeOH, 1.45 h; Step C: 4M HCl, 1,4-dioxane, rt, 2 h; Step D: 3-benzyloxyformylamino-(2R)-cyclopentylmethyl propionic acid, HATU, collidine, DCM, 0° C., 15 min, then hydrazine, 0° C., 1 h; Step E: benzyl 2-bromoacetate, DIEA, CH₂Cl₂, 45° C., 16 h; Step F: H₂ (g), 10% Pd/C, MeOH, rt, 2 h; Step G: EDAC, HOAt, DCM, 0° C., 15 min, then 4-benzyl piperidine, 0° C. to rt, 16 h.

Step A:

### Isopropylidine hydrazine

To a solution of tert-butyl carbazate (2.64 g, 20 mmol) in THF (50 ml) at rt under argon was added acetone (1.61 ml, 22 mmol) and sodium triacetoxyborohydride (4.45 g, 22 mmol). The resulting suspension was stirred for 16 h. After this time the mixture was concentrated under reduced pressure and the crude product was partitioned between EtOAc and sat. NaHCO₃. The organic layer was separated, washed with sat. NaCl, dried (MgSO₄), filtered and concentrated under reduced pressure to give a pale yellow oil. This oil was purified by chromatography on silica eluting with 50% EtOAc/hexanes to give the title compound as a white solid (2.95 g, 85%). 1H-NMR; δ(CDCl₃), 7.34 (1H, s, NH), 2.04 (3H, s, CH₃), 1.81 (3H, s, CH₃), 1.81 (3H, s, CH₃), 1.51 (9H, s, (CH₃)₃). LRMS: +ve ion: 195 [M+23], 117.

Step B:

### N′-(Isopropyl)hydrazine carboxylic acid tert-butyl ester

To a solution of isopropylidine hydrazine (2.94 g, 17.1 mmol) in MeOH at rt under argon was added 5% PVC (290 mg). Hydrogen gas was bubbled through the black suspension for 15 min and then the reaction was left under an atmosphere of hydrogen for 1.5 h. After this time, the suspension was filtered and the collected solids were washed with additional MeOH. The combined filtrate and washings were concentrated under reduced pressure. The crude material that remained was then redissolved in EtOAc and washed with sat. NaHCO₃. The organic layer was separated, washed with sat. NaCl, dried (MgSO₄), filtered and concentrated under reduced pressure. Purification by chromatography on silica eluting with 50% EtOAc/hexanes gave the title compound as colourless crystals (2.08 g, 70%). 1H-NMR; δ (CDCl₃), 6.34 (1H, s NHBoc), 3.50 (1H, br, NH) 3.13 (1H, sept, J=6.5 Hz, CH(CH₃)₂), 1.46 (9H, s, (CH₃)₃), 1.02 (6H, d, J=6.5 Hz, (CH₃)₂CH). LRMS: +ve ion: 197 [M+23], 175 [M+1], 161.

Step C:

### Isopropyl hydrazine hydrochloride

To N′-(Isopropyl)-hydrazine carboxylic acid tert-butyl ester (1.26 g, 7.24 mmol) at rt in air was added 4M HCl in 1,4-dioxane (25 ml). The mixture was stirred for 2 h and then concentrated under reduced pressure to give a white solid which was used in the next step (assumed quantative yield, 7.24 mmol).

Step D:

### N-Benzyloxy-N-[3-cyclopentyl-(2R)-(N′-isopropylhydrazinocarbonyl)-propyl]-formamide

To a solution of the 3-benzyloxyformylamino-(2R)-cyclopentylmethyl propionic acid (190 mg, 0.62 mmol) in DCM (5 ml) at 0° C. under argon was added HATU (237 mg, 0.62 mmol) followed by collidine (165 μl, 2.49 mmol). The resulting mixture was stirred for 5 min at rt before the amine hydrochloride (69 mg, 0.62 mmol) was added as a solution in DCM (1 ml) and collidine (165 μl, 2.49 mmol). The reaction was stirred at 0° C. for 1 h and then the mixture was concentrated under reduced pressure. The crude material was partitioned between EtOAc and sat. NaHCO₃. The organics were separated, washed with sat. NaCl, dried (MgSO₄), filtered and concentrated under reduced pressure to give an oil. This oil was purified by chromatography on silica eluting with 70-100% EtOAc/hexanes to give the title compound as a colourless oil (119 mg, 53%). 1H-NMR; δ(CDCl₃), 8.12 (0.67H, s, CHO), 7.86 (0.33H, br s, CHO), 7.5-7.3 (6H, br m, ArH and NH), 4.9-4.6 (2H, br m, OCH₂Ph), 3.8-3.6 (2H, br m, CH₂NO), 3.01 (1H, sept, J=6.5 Hz, CH(CH₃)₂), 2.5-2.4 (1H, m, CHCO), 1.8-1.3 (9H, m), 1.2-0.9 (2H, m), 0.98 (6H, d, J=6.5 Hz, (CH₃)₂CH). LRMS: +ve ion: 362 [M+1].

Step E:

### {N′-[3-(Benzyloxy-formyl-amino)-(2R)-cyclopentylmethyl-propionyl]-N-isopropyl-hydrazino}acetic acid benzyl ester

To N-benzyloxy-N-[3-cyclopentyl-(2R)-(N′-isopropylhydrazinocarbonyl)-propyl]-formamide (69 mg, 0.191 mmol) in DCM (2.5 ml) under argon was added benzyl 2-bromoacetate (30 μl, 0.19 mmol) followed by DIEA (33 μl, 0.19 mmol). The reaction was stirred at 45° C. for 16 h. After this time, methylisocyanate polystyrene resin (198 mg, 0.09 mmol)(200-400 mesh, 1.45 mmol/g) was added and the mixture shaken for 12 h. The mixture was filtered and concentrated under reduced pressure to give a crude product which was purified by chromatography on silica eluting with EtOAc to give the title compound as a colourless oil (32 mg, 33%). 1H-NMR; δ(CDCl₃), 8.1-7.8 (1H, m, CHO), 7.6-7.2 (10H, m, 2×Ph), 5.3-5.0 (2H, m, NOCH₂), 5.0-4.6 (2H, m, NCH₂CO), 3.8-3.5 (4H, m, CH₂NO and CO₂CH₂), 3.3-3.1 (1H, m, CH(CH₃)₂), 3.1-2.4 (1H, br m, COCHCH₂), 1.9-0.8 (11H, m), 1.04 (6H, d, J=6.5 Hz, CH(CH₃)₂); LRMS: +ve ion: 532 [M+23], 510 [M+1].

Step F:

### {N′-[(2R)-Cyclopentylmethyl-3-(formyl-hydroxy-amino)-propionyl]-N-isopropyl-hydrazino}acetic acid

To a solution of the {N′-[3-(benzyloxy-formyl-amino)-(2R)-cyclopentylmethyl-propionyl]-N-isopropyl-hydrazino}acetic acid benzyl ester (31 mg, 0.061 mmol) in EtOH at rt under argon was added 10% Pd/C (5 mg). Hydrogen gas was bubbled through the black suspension for 15 min and then the reaction was left under an atmosphere of hydrogen for 1 h. After this time, the suspension was filtered and the collected solids were washed with additional MeOH. The combined filtrate and washings were concentrated under reduced pressure to give a colourless oil (18 mg, 92%). 1H-NMR; δ (CD₃OD), 8.23 (0.3H, s, CHO), 7.84 (0.7H, s, CHO), 3.9-2.6 (6H, m), 1.9-0.9 (17H, m). LRMS: +ve ion: 352 [M+23], 330 [M+1].

Step G:

### N-(2-{N′-[(2R)-(4-Benzyl-piperidin-1-yl)-2-oxo-ethyl]-N′-isopropyl-hydrazinocarbonyl}-3-cyclopentyl propyl)-N-hydroxy formamide

To a solution of {N′-[(2R)-cyclopentylmethyl-3-(formyl-hydroxy-aminoypropionyl]-N-isopropyl-hydrazino}acetic acid (18 mg, 0.055 mmol) in DMF (2 ml) at 0° C. under argon was added EDAC (13 mg, 0.066 mmol) and HOAt (11 mg, 0.083 mmol). The resulting mixture was stirred for 15 min before 4-benzylpiperidine (11 μl, 0.083 mmol) was added. The reaction was stirred for 15 min at 0° C. and then warmed to rt where it was maintained for 16 h. The reaction mixture was diluted with EtOAc and washed with water. The aqueous layer was extracted with EtOAc (x2) and the combined organics washed with sat. NaCl, dried (MgSO₄), filtered and concentrated under reduced pressure to give an oil. Purification by preparative HPLC gave the title compound as a colourless oil (6 mg, 22%). 1H-NMR; δ(CD₃OD), 8.23 (0.33H, s, CHO), 7.84 (0.67H, s, CHO), 7.3-7.0 (5H, m, Ph), 4.5-4.0 (2H, m, CH₂CO), 3.8-2.6 (8H, m), 2.6-2.5 (2H, m, CH₂Ph), 1.9-0.9 (22H, m). LRMS: +ve ion: 362 [M+1].

### EXAMPLE 2

Prepared According to Reaction Scheme 1

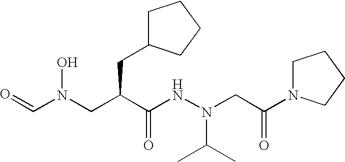

### N-{3-Cyclopentyl-(2R)-N′-isopropyl-N′-(2-oxo-2-pyrrolidin-1-yl-ethyl)-hydrazinocarbonyl]-propyl)N-hydroxy-formamide

Clear colourless oil (28 mg, 48%). 1H-NMR; δ(CD₃OD), 8.23 (0.4H, s, CHO), 7.84 (0.6H, s, CHO), 3.9-3.1 (9H, m), 2.9-2.6 (1H, m, NCHMe₂), 2.1-1.0 (15H, m), 1.06 (6H, d, J 6.5, CHMe₂). LRMS: +ve ion: 405 [M+23], 383 [M+1].

### EXAMPLE 3

Prepared According to Reaction Scheme 1

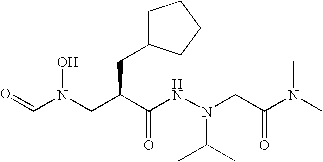

### 2-{N′-[(2R)-Cyclopentylmethyl-3-(formyl-hydroxy-amino)-propionyl]-N-isopropyl-hydrazino}-N,N-dimethyl-acetamide

Clear colourless oil (27 mg, 50%). 1H-NMR; δ(CD₃OD), 8.24 (0.3H, s, CHO), 7.84 (0.7H, s, CHO), 3.8-2.4 (6H, m), 3.14 (3H, s, NMe), 2.88 (3H, s, NMe), 2.0-0.9 (17H, m). LRMS: +ve ion: 379 [M+23], 357 [M+1].

### EXAMPLE 4

Prepared According to Reaction Scheme 1

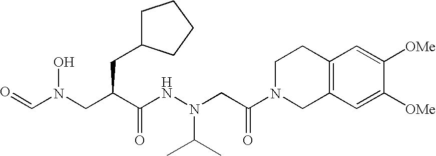

### N-{3-Cyclopentyl-(2R)-[N′-[2-(6,7-dimethoxy-3,4-dihydro-1H-isoquinolin-2-yl)-2-oxo-ethyl]-N′-isopropyl-hydrazinocarbonyl}-propyl)-N-hydroxy-formamide

Clear colourless oil (6 mg, 8%). 1H-NMR; δ(CD₃OD), 8.2-7.8 (1H, m, CHO), 6.8-6.7 (2H, m, ArH), 4.8-4.4 (2H, m), 4.0-2.5 (16H, m), 1.9-1.0 (17H, m). LRMS: +ve ion: 527 [M+Na], 505 [M+1].

### EXAMPLE 5

Prepared According to Reaction Scheme 1

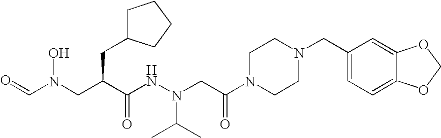

### N-(2-{N′-[(2R)-(4-Benzo[1,3]dioxol-5-ylmethyl-piperazin-1-yl)-2-oxo-ethyl]-N′-isopropyl-hydrazinocarbonyl}-3-cyclopentyl propyl)-N-hydroxy formamide

colourless oil (34 mg, 42%). 1H-NMR; δ(CD₃OD), 1H-NMR; 3(CD₃OD), 8.23 (0.3H, s, CHO), 7.83 (0.7H, s, CHO), 6.9-6.7 (3H, m, ArH), 5.92 (2H, s, OCH₂O), 3.9-2.2 (16H, m), 2.0-0.9 (17H, m). LRMS: +ve ion: 332 [M+1].

### EXAMPLE 6

Prepared According to Reaction Scheme 1

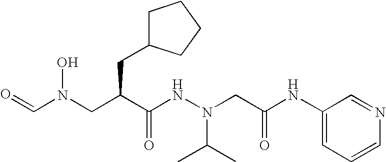

### 2-N′-[(2R)-Cyclopentylmethyl-3-(formyl-hydroxy-amino)propionyl]-N-isopropyl-hydrazino}-N-pyridin-3-yl-acetamide

colourless oil (20 mg, 32%). 1H-NMR; δ(CD₃OD), 1H-NMR; δ(CD₃OD), 8.87 (1H, dd, J 7.5 & 2, ArH), 8.25 (1H, d, J 5, ArH), 8.20 (0.45H, s, CHO), 8.2-8.1 (1H, m, ArH), 7.80 (0.55H, s, CHO), 7.40 (1H, dd, J 8.5 &5, ArH), 3.8-3.1 (5H, m), 3.0-2.6 (1H, m, NCHMe₂), 1.9-0.7 (17H, m). LRMS: +ve ion: 428 [M+23], 406 [M+1].

### EXAMPLE 7

Prepared According to Reaction Scheme 1

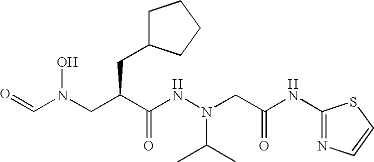

### 2-{N′-[(2R)-Cyclopentylmethyl-3-(formyl-hydroxy-amino)-propionyl]-N-isopropyl-hydrazino}-N-thiazol-2-yl-acetamide

colourless oil (28 mg, 44%). 1H-NMR; δ(CD₃OD), 1H-NMR; δ(CD₃OD), 8.19 (0.3H, s, CHO), 7.79 (0.7H, s, CHO), 7.44 (1H, d, J 3.5, ArH), 7.12 (1H, d, J 3.5, ArH), 3.9-3.1 (5H, m), 2.9-2.5 (1H, m, NCHMe₂), 1.9-0.8 (17H, m). LRMS: +ve ion: 434 [M+23], 412 [M+1].

### EXAMPLE 8

Prepared According to Reaction Scheme 1

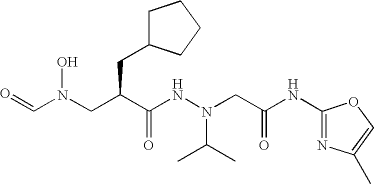

### 2-{N′-[(2R)-Cyclopentylmethyl-3-(formyl-hydroxy-amino)-propionyl]-N-isopropyl-hydrazino}-N-(4-methyl-oxazol-2-yl)-acetamide

colourless oil (6 mg, 10%). 1H-NMR; δ(CD₃OD), 1H-NMR; δ(CD₃OD), 8.20 (0.3H, s, CHO), 7.83 (0.7H, s, CHO), 7.33 (1H, s, ArH), 3.8-3.1 (5H, m), 2.9-2.6 (1H, m, NCHMe₂), 2.10 (3H, s, ArMe), 2.0-1.0 (17H, m). LRMS: +ve ion: 432 [M+23], 410 [M+1].

### EXAMPLE 9

Prepared According to Reaction Scheme 1

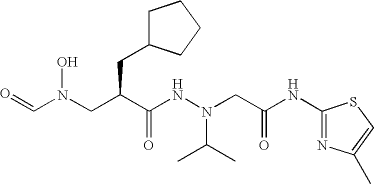

### 2-{N′-[(2R)-Cyclopentylmethyl-3-(formyl-hydroxy-amino)-propionyl]-N-isopropyl-hydrazino}-N-(4-methyl-thiazol-2-yl)-acetamide

colourless oil (18 mg, 28%). 1H-NMR; δ(CD₃OD), 1H-NMR; δ(CD₃OD), 8.19 (0.3H, s, CHO), 7.80 (0.7H, s, CHO), 6.66 (1H, s, ArH), 3.8-3.1 (5H, m), 2.9-2.5 (1H, m, NCHMe₂), 2.30 (3H, s, ArMe), 1.9-0.8 (17H, m). LRMS: +ve ion: 448 [M+23], 426 [M+1].

### EXAMPLE 10

Prepared According to Reaction Scheme 1

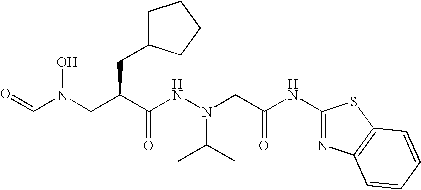

### N-Benzothiazol-2-yl-2-{N′-[(2R)-cyclopentylmethyl-3-(formyl-hydroxy-amino)-propionyl]-N-isopropyl-hydrazino)acetamide

colourless oil (46 mg, 66%). 1H-NMR; δ(CD₃OD), 1H-NMR; δ(CD₃OD), 8.19 (0.3H, s, CHO), 7.85 (1H, d, J 8, ArH), 7.80 (0.7H, s, CHO), 7.77 (1H, d, J 8, ArH), 7.43 (1H, t, J 8, ArH), 7.30 (1H, t, J 8, ArH), 3.9-3.1 (5H, m), 2.9-2.6 (1H, m, NCHMe₂), 1.9-0.8 (17H, m). LRMS: +ve ion: 484 [M+23], 462 [M+1].

### EXAMPLE 11

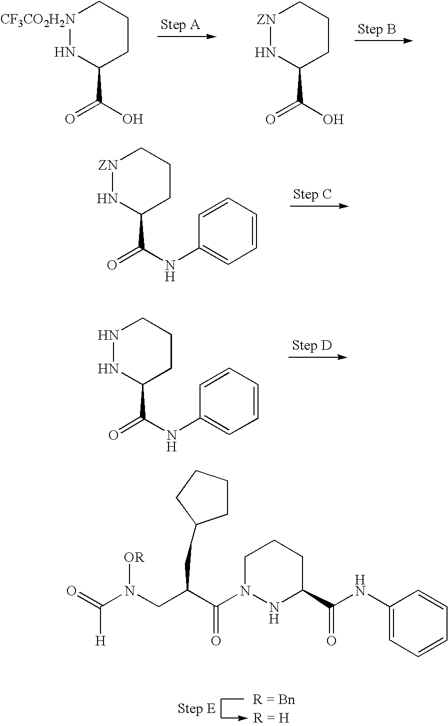

Step A: PhCH₂OCOCl, 1M NaOH (aq), PhMe, 0° C.; Step B: EDAC.HCl, HOAt, aniline, DMF 0° C. to rt; Step C: H₂, 10% Pd/C, EtOH; Step D: 3-benzyloxyformylamino-(2R)-cyclopentylmethyl propionic acid, EDAC.HCl, HOAt, 0° C., 15 min, then piperazic acid amide, 0° C. to rt; Step E: H₂ (9), 10% Pd/C, EtOH, rt, 2 h.

Step A:

### Tetrahydro-pyridazine-(1,3S)-dicarboxylic acid 1-benzyl ester

To a solution of (3S)-piperazic acid trifluoroacetic acid salt (129 mg, 0.53 mmol) in toluene/l M NaOH (1:1, 4 ml) at rt under argon was added benzyl chloroformate (114 μl, 0.79 mmol). The resulting solution was stirred for 1 h. After this time the mixture was diluted with water, washed with Et₂O (x3) and acidified to pH 5 with 1M HCl. The aqueous fraction was then extracted with EtOAc (x3), dried (MgSO₄), filtered and concentrated under reduced pressure to give a crude product as a white solid (125 mg, 89%). 1H-NMR; δ(CD₃OD), 7.5-7.2 (5H, m, Ph), 5.12 (2H, s, CH₂Ph), 4.0-3.8 (1H, m, NCH_{A}H_{B}), 3.6-3.5 (1H, m, NCHCO₂), 3.3-3.1 (1H, m, NCH_{A}H_{B}), 2.1-2.0 (1H, m), 1.8-1.5 (3H, m). LRMS: +ve ion: 287 [M+23], 265 [M+1].

Step B:

(3S)-Phenylcarbamoyl-tetrahydro-pyridazine-1-carboxylic acid benzyl ester To a solution of tetrahydro-pyridazine-(1,3S)-dicarboxylic acid 1-benzyl ester (85 mg, 0.32 mmol) in DMF (2 ml) at 0° C. under argon was added EDAC (74 mg, 0.39 mmol) and HOAt (66 mg, 0.48 mmol). The resulting mixture was stirred for 15 min before aniline (32 μL, 0.35 mmol) was added. The reaction was stirred for 15 min at 0° C. and then warmed to rt where it was maintained for 16 h. The reaction mixture was diluted with EtOAc and washed with water. The aqueous layer was extracted with EtOAc (x2) and the combined organics washed with sat. NaHCO₃, sat. NaCl, dried (MgSO₄), filtered and concentrated under reduced pressure to give a crude product as an oil. Purification by chromatography on silica eluting with 30% EtOAc/hexane gave the title compound as a colourless oil (83 mg, 76%). 1H-NMR; δ(CD₃OD), 7.7-7.4 (2H, br s, ArH), 7.4-7.2 (7H, m, ArH), 7.07 (1H, t, J=7.5 Hz, ArH), 5.20 (1H, d, J=5 Hz, OCH_{A}H_{B}Ph), 5.11 (1H, d, J=5 Hz, OCH_{A}H_{B}Ph), 3.82 (1H, dt, J=13 and 5 Hz, NCH_{A}H_{B}), 3.69 (1H, t, J=5 Hz, NCHCO), 3.4-3.2 (1H, m, NCH_{A}H_{B}), 2.3-2.1 (1H, m), 1.9-1.4 (3H, m). LRMS: +ve ion: 362 [M+23].

Step C:

### Hexahydro-pyridazine-(3S)-carboxylic acid phenylamide

To a solution of the (3S)-phenylcarbamoyl-tetrahydro-pyridazine-1-carboxylic acid benzyl ester (83 mg, 0.25 mmol) in EtOH at rt under argon was added 10% Pd/C (8 mg). Hydrogen gas was bubbled through the black suspension for 15 min and then the reaction was left under an atmosphere of hydrogen for 1 h. After this time, the suspension was filtered and the collected solids were washed with additional MeOH. The combined filtrate and washings were concentrated under reduced pressure to give a colourless oil (49 mg, 98%) which was used immediately for the next step. LRMS: +ve ion: 228 [M+23], 206 [M+1].

Step D:

### 1-{(2R)-(Benzyloxy-formyl-amino)-methyl]-3-cyclopentyl-propionyl}-hexahydro-pyridazine-(3S)-carboxylic acid phenylamide

To a solution of 3-benzyloxyformylamino-(2R)-cyclopentylmethyl propionic acid (44 mg, 0.15 mmol) in DMF (3 ml) at 0° C. under argon was added EDAC (30 mg, 0.16 mmol) and HOAt (27 mg, 0.19 mmol). The resulting mixture was stirred for 15 min before a solution of hexahydro-pyridazine-(3S)-carboxylic acid phenylamide (27 mg, 0.13 mmol) in DMF (0.5 ml) was added. The reaction was stirred for 15 min at 0° C. and then warmed to rt where it was maintained for 16 h. The reaction mixture was diluted with EtOAc and washed with water. The aqueous layer was extracted with EtOAc (x2) and the combined organics washed with sat. NaCI, dried (MgSO₄), filtered and concentrated under reduced pressure to give an oil. This oil was purified by chromatography on silica eluting with EtOAc to give the title compound as a colourless oil (18 mg, 25%). 1H-NMR; δ(CDCl₃), 9.56 (1H, brs, NH), 8.4-7.9 (1H, m, CHO), 7.8-7.0 (10H, m, ArH), 5.1-4.5 (3H, m), 4.0-3.0 (5H, m), 2.5-0.9 (15H, m). LRMS: +ve ion: 515 [M+23].

Step E:

### 1-[(2R)-Cyclopentylmethyl-3-(formyl-hydroxy-amino)-propionyl]-hexahydro-pyridazine-(3S)-carboxylic acid phenylamide

To a solution of the 1-{(2R)-(benzyloxy-formyl-amino)-methyl]-3-cyclopentyl-propionyl}-hexahydro-pyridazine-(3S)-carboxylic acid phenylamide (18 mg, 0.037 mmol) in EtOH (2 ml) at rt under argon was added 10% Pd/C (4 mg). Hydrogen gas was bubbled through the black suspension for 15 min and then the reaction was left under an atmosphere of hydrogen for 1 h. After this time, the suspension was filtered and the collected solids were washed with additional MeOH. The combined filtrate and washings were concentrated under reduced pressure to give a colourless oil. This oil was purified by preparative HPLC to give the title compound as a colourless oil (8 mg, 50%). 1H-NMR; δ (CD₃OD), 8.26 (0.4H, s, CHO), 7.85 (0.6H, s, CHO), 7.7-7.5 (2H, m, ArH), 7.29 (2H, t, J=7.5 Hz, ArH), 7.11 (1H, t, J=7.5 Hz, ArH), 4.5-4.4 (1H, m, NCHCON), 4.2-3.2 (4H, m), 2.9-2.7 (1H, m), 2.2-0.8 (15H, m). LRMS: +ve ion: 425 [M+23], 403 [M+1].

### Biological Example

Minimal Inhibitory concentrations (MIC) of inhibitors against clinical isolates of S. pneumoniae, H. influenzae and M. catarrhalis obtained from the Public Health and Clinical Microbiology Laboratory, Addenbrooke's Hospital, Hills Road, Cambridge CB2 2QW, UK, were determined by a standard agar plate dilution method following recommendations in British Society for Antimicrobial Chemotherapy Working Party. 1991 “A guide to sensitivity testing British Society for Antimicrobial Chemomtherapy, London, United Kingdom”. Briefly Iso-Sensitest agar (pH 7.2: Oxoid, United Kingdom) was employed supplemented with 5% horse blood (Oxoid) and 20 μg of NAD (Sigma) per ml to support growth of fastidious bacteria. The inoculum used was approximately 10⁴ colony forming units of each isolate contained in a volume of 1 μl. Plate were incubated 18 to 24 hr in air, -or for fastidious bacteria an atmosphere enriched with 4-6% carbon dioxide at 35° C. The MIC was determined as the lowest concentration of an antimicrobial tested that inhibited growth of the inoculum, disregarding a single persisting colony or faint haze caused by the inoculation.

The compounds of the Examples were are antibacterially active in the above assays, The following table states the MIC ranges of the tested compounds against 3 strains of S. pneumoniae, 2 strains of H. influenzae and 1 strain of M. catarrhalis.

| | S. pneumoniae | H. influenzae | M. catarrhalis |
|---|---|---|---|
| Example | μg/ml | μg/ml | μg/ml |
| 2 | 4-16 | 8 | 0.5 |
| 3 | 16−>32 | 0.5-1 | 0.5 |
| 4 | 1-2 | 32 | 0.5 |
| 5 | 1-4 | >32 | 1 |
| 6 | 32−>32 | 32 | 2 |
| 7 | 16-32 | 16-32 | 2 |
| 8 | 8-16 | 8−>32 | 2 |
| 9 | 16-32 | >32 | 2 |
| 10 | 1-4 | >32 | 2 |

1. A compound of formula (I) or a pharmaceutical or veterinary acceptable salt, hydrate or solvate thereof 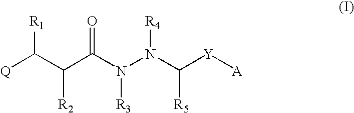
wherein
Q represents a radical of formula —N(OH)CH(═O) or formula —C(═O)NH(OH);
Y represents —C(═O)—, —C(═S)—, —S(═O)—, or —SO₂—;
R₁ represents hydrogen, C₁-C₆ alkyl or C₁-C₆ alkyl substituted by one or more halogen atoms, or, except when Q is a radical of formula —N(OH)CH(═O), a hydroxy, C₁-C₆ alkoxy, C₁-C₆ alkenyloxy, halogen, amino, C₁-C₆ alkylamino, or di-(C₁-C₆ alkyl)amino group;
R₂ represents a substituted or unsubstituted C₁-C₆ alkyl, C₁-C₃ alkyl-O—C₁-C₃ alkyl, C₁-C₃ alkyl-S—C₁-C₃ alkyl, cycloalkyl(C₁-C₃ alkyl)-, aryl(C₁-C₃ alkyl)-, heterocyclyl(C₁-C₃ alkyl)-, or R¹R²N—C₁-C₃ alkyl group wherein R¹ represents hydrogen or C₁-C₃ alkyl and R² represents C₁-C₃ alkyl, or R¹ R²N-represents a cyclic amino group;
R₃ and R₅ independently represent hydrogen or a substituted or unsubstituted C₁-C₆ alkyl group or R₃ and R₅ taken together with the carbon and nitrogen atoms to which they are respectively attached form a saturated heterocyclic ring of from 5 to 7 ring atoms, which may be fused to a second carbocyclic or heterocyclic ring, either of which rings may optionally be substituted;
R₄ represent hydrogen or a substituted or unsubstituted C₁-C₆ alkyl, C₂-C₆ alkenyl, C₂-C₆ alkynyl, cycloalkyl, aryl, heterocyclyl, C₁-C₃ alkyl-O—C₁-C₃ alkyl, C₁-C₃-alkyl-S—C₁-C₃ alkyl, C₁-C₃ alkyl-NH—(C₁-C₃ alkyl)-, cycloalkyl(C₁-C₃ alkyl)-, heterocyclic(C₁-C₃ alkyl)- or aryl(C₁-C₃ alkyl)- group; and
A represents a primary, secondary or tertiary amino group or a group- R₆, —OR₆, wherein R₆ is a substituted or unsubstituted C₁-C₆ alkyl, C₂-C₆ alkenyl, C₂-C₆ alkynyl, cycloalkyl, aryl, heterocyclyl, C₁-C₃ alkyl-O—(C₁-C₃ alkyl), C₁-C₃ alkyl-S—(C₁-C₃ alkyl)-, C₁-C₃ alkyl-NH—(C₁-C₃ alkyl)-, cycloalkyl(C₁-C₃ alkyl)-, heterocyclic(C₁-C₃ alkyl)-1 or aryl(C₁-C₃ alkyl)-group.

2. A compound as claimed in claim 1 wherein Q is an N-formyl hydroxylamine group —N(OH)CH(═O).

3. A compound as claimed in claim 1 wherein —Y— is —C(═O)— or —SO₂—.

4. A compound as claimed in claim 1 wherein R₁ is hydrogen.

5. A compound as claimed in claim 1 wherein R₂ is
optionally substituted C₁-C₆ alkyl, C₃-C₆ alkenyl, C₃-C₆ alkynyl or cycloalkyl;
phenyl(C₁-C₆ alkyl)-, phenyl(C₃-C₆ alkenyl)- or phenyl(C₃-C₆ alkynyl)- optionally substituted in the phenyl ring;
cycloalkyl(C₁-C₆ alkyl)-, cycloalkyl(C₃-C₆ alkenyl)- or cycloalkyl(C₃-C₆ alkynyl)- optionally substituted in the cycloalkyl ring; or
CH₃(CH₂)ₚO(CH₂)_{q}— or CH₃(CH₂)ₚS(CH₂)_{q}—, wherein p is 0, 1, 2 or 3 and q is 1, 2 or 3.

6. A compound as claimed in claim 1 wherein R₂ is methyl, ethyl, n- or iso-propyl, n-or iso-butyl, n-pentyl, iso-pentyl, 3-methyl-but-1-yl, n-hexyl, n-heptyl, n-acetyl, n-octyl, methylsulfanylethyl, ethylsulfanylmethyl, 2-methoxyethyl, 2-ethoxyethyl, 2-ethoxymethyl, 3-hydroxypropyl, allyl, 3-phenylprop-3-en-1-yl, prop-2-yn-1-yl, 3-phenylprop-2-yn- 1 -yl, 3-(2-chlorophenyl)prop-2-yn- 1 -yl, but-2-yn-1-yl, cyclopentyl, cyclohexyl, cyclopentylmethyl, cyclopentylethyl, cyclopentylpropyl, acyclohexylmethyl, cyclohexylethyl, cyclohexylpropyl, furan-2-ylmethyl, furan-3-methyl, tetrahydrofuran-2-ylmethyl, tetrahydrofuran-2-ylmethyl, piperidinylmethyl, pyrid-2-ylmethyl, pyrid-3-ylmethyl, pyrid-4-ylmethyl, phenylpropyl, 4-chlorophenylpropyl, 4-methylphenylpropyl, 4-methoxyphenylpropyl, benzyl, 4-chlorobenzyl, 4-methylbenzyl, or 4-methoxybenzyl.

7. A compound as claimed in claim 1 wherein R₂ is (C₁-C₆)alkyl-, cycloalkylmethyl-, (C₁-C₃)alkyl-S—(C₁-C₃)alkyl-, or (C₁-C₃)alkyl-O—(C₁-C₃)alkyl-.

8. A compound as claimed in claim 7 wherein R₂ is a n-propyl, n-butyl, n-pentyl, cyclopentylmethyl, cyclopentylethyl, cyclohexylmethyl or cyclohexylethyl.

9. A compound as claimed claim 1 wherein R₄ is hydrogen, (C₁-C₆)alkyl-, cycloalkylmethyl-, (C₁-C₃)alkyl-S—(C₁-C₃)alkyl-, or (C₁-C₃)alkyl-O—(C₁-C₃)alkyl-.

10. A compound as claimed in claim 9 wherein R₄ is hydrogen, methyl, ethyl, n-propyl, n-butyl, n-pentyl, cyclopentylmethyl, cyclopentylethyl, cyclohexylmethyl or cyclohexylethyl.

11. A compound as claimed in claim 1 wherein R₃ and R₅, when not part of a ring, are independently hydrogen, (C₁-C₆)alkyl-, cycloalkylmethyl-, (C₁-C₃)alkyl-S—(C₁-C₃)alkyl-, or (C₁-C₃)alkyl-O—(C₁-C₃)alkyl-.

12. A compound as claimed in claim 11 wherein R₃ and R₅ are independently hydrogen, methyl, ethyl, n-propyl, n-butyl, n-pentyl, cyclopentylmethyl, cyclopentylethyl, cyclohexylmethyl or cyclohexylethyl.

13. A compound as claimed in claim 1 wherein R₃ and R₅ taken together with the carbon and nitrogen atoms to which they are respectively attached form the following rings, wherein any sulfur atom present as a ring member may be oxidized to —SO— or —SO₂—, R₄ is as defined in claim 1, and R represents hydrogen or C₁-C₄ alkyl:

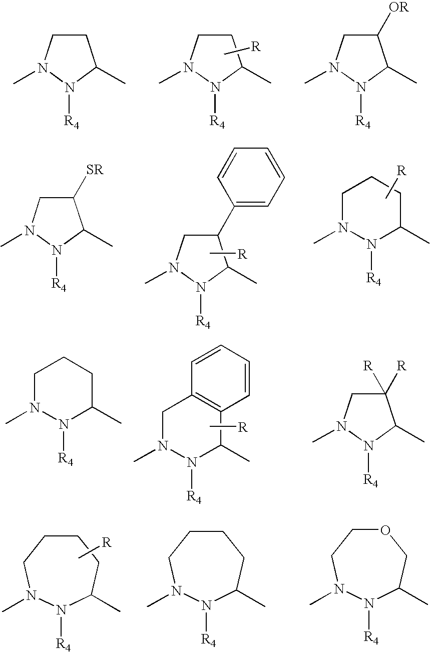

14. A compound as claimed in claim 1 wherein A is a group —NR₆R₇, —R₆, or —OR₆ wherein R₆ and R₇ independently represent a radical of formula (IV) 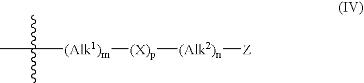
wherein
m, p and n are independently 0 or 1;
Z represents hydrogen or a carbocyclic or heterocyclic ring of 5 to 7 ring atoms which is optionally fused to a saturated or unsaturated carbocyclic or heterocyclic second ring of 5 to 7 ring atoms;
Alk¹ and Alk² independently represent divalent C₁-C₃ alkylene radicals;
X represents —O—, —S—, —S(O)—, —S(O₂)—, —C(═O)—, —NH—, —NR₇— where R₇ is C₁-C₃ alkyl;
and wherein
Alk¹ and Alk² and Z when other than hydrogen, independently are optionally substituted by
(C₁-C₃)alkyl, (C₂-C₃)alkenyl, or (C₂-C₃)alkynyl,
phenyl, optionally substituted by (C₁-C₃)alkyl, (C₁-C₃)alkoxy, halo, nitro, amino, mono- or di-(C₁-C₃3) alkylamino, cyano or trifluoromethyl;
monocyclic 5 or 6-membered heterocyclic, optionally substituted by(C₁-C₃)alkyl, (C₁-C₃)alkoxy, halo, nitro, amino, mono- or di-(C₁-C₃)alkylamino, cyano or trifluoromethyl
benzyl, optionally substituted in the phenyl ring by (C₁-C₃)alkyl, (C₁-C₃)alkoxy, halo, nitro, amino, mono- or di-(C₁-C₃)alkylamino, cyano or trifluoromethyl,
hydroxy, phenoxy, (C₁-C₆)alkoxy, or hydroxyl (C₁-C₆)alkyl,
mercapto, (C₁-C₆) alkylthio or mercapto (C₁-C₆)alkyl,
oxo,
nitro,
cyano
halo
—COOH, or —COOR^{A},
—COONH₂, —CONHR^{A}, or —CONR^{A}R^{B}
—COR^{A}, —SO₂R^{A},
—NHCOR^{A},
—NH₂, —NHR^{A}, or —NR^{A}R^{B},
wherein R^{A} and R^{B} are independently a (C₁-C₆) alkyl group, R^{A} and R^{B} taken together with the nitrogen atom to which they are attached form a 5- or 6-membered heterocyclic ring which may be substituted by (C₁-C₃)alkyl, hydroxyl, or hydroxyl(C₁-C₃)alkyl.

15. A compound as claimed claim 1 wherein A is a group —NR₈R₉ wherein R₈ and R₉ when taken together with the nitrogen atom to which they are attached form a saturated heterocyclic ring of 5 to 8 atoms optionally fused to a saturated or unsaturated carbocyclic or hetercyclic second ring of 5 to 7 ring atoms, any of which rings being optionally substituted by a radical of formula (IV) 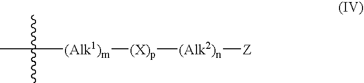
wherein
m, p and n are independently 0 or 1;
Z represents hydrogen or a carbocyclic or heterocyclic ring of 5 to 7 ring atoms which is optionally fused to a saturated or unsaturated carbocyclic or heterocyclic second ring of 5 to 7 ring atoms,
Alk¹ and Alk² independently represent divalent C₁-C₃ alkylene radicals;
X represents —O—, —S—, —S(O)—, —S(O)—, —C(═O)—, —NH—, —NR₇— where R₇ is C₁-C₃ alkyl;
and wherein
Alk¹ and Alk² and Z when other than hydrogen, independently are optionally substituted by
(C₁-C₃)alkyl, (C₂-C₃)alkenyl, or (C₂-C₃)alkynyl,
phenyl, optionally substituted by (C₁-C₃)alkyl, (C₁-C₃)alkoxy, halo, nitro, amino, mono- or di-(C₁-C₃)alkylamino, cyano or trifluoromethyl;
monocyclic 5 or 6-membered heterocyclic, optionally substituted by(C₁-C₃)alkyl, (C,₁-C₃)alkoxy, halo, nitro, amino, mono- or di-(C₁-C₃)alkylamino, cyano or trifluoromethyl
benzyl, optionally substituted in the phenyl ring by (C₁-C₃)alkyl, (C₁-C₃)alkoxy, halo, nitro, amino, mono- or di-(C₁-C₃)alkylamino, cyano or trifluoromethyl,
hydroxy, phenoxy, (C₁-C₆)alkoxy, or hydroxyl (C₁-C₆)alkyl,
mercapto, (C₁-C₆) alkylthio or mercapto (C₁-C₆)alkyl,
oxo,
nitro,
cyano
halo
—COOH, or —COOR^{A},
—COONH₂, —CONHR^{A}, or —CONR^{A}R^{B}
—COR , —SO₂R^{A}
—NHCOR^{A},
—NH₂, —NHR^{A}, or —NR^{A}R^{B},
wherein R^{A} and R^{B} are independently a (C₁-C₆) alkyl group, R^{A} and R^{B} taken together with the nitrogen atom to which they are attached form a 5- or 6-membered heterocyclic ring which may be substituted by (C₁-C₃)alkyl, hydroxyl, or hydroxyl(C₁-C₃)alkyl.

16. A compound as claimed in claim 15 wherein R₈ and R₉ when taken together with the nitrogen atom to which they are attached form an optionally substituted 1-pyrrolidinyl, piperidin-1-yl, 1-piperazinyl, hexahydro-1-pyridazinyl, morpholin-4-yl, tetrahydro-1,4-thiazin-4-yl, tetrahydro-1,4-thiazin-4-yl 1-oxide, tetrahydro-1,4-thiazin-4-yl, 1,1-dioxide, hexahydroazipino, thiomorpholino, diazepino, or thiazolidinyl ring.

17. A compound as claimed in claim 15 wherein R₈ and R₉ when taken together with the nitrogen atom to which they are attached form an optionally substituted piperidin-1-yl or 1-piperazinyl ring.

18. A compound as claimed in claim 14 wherein in the group (IV) Alk¹ and Alk² independently represent —(CH₂)— or —CH₂CH₂)—.

19. A compound as claimed in claim 14 wherein in the group (IV) m is 0, p is 1, and X is —C(═O)— or —S(O₂)—.

20. A compound as claimed in claim 1 of formulae (IIA)-(IID). 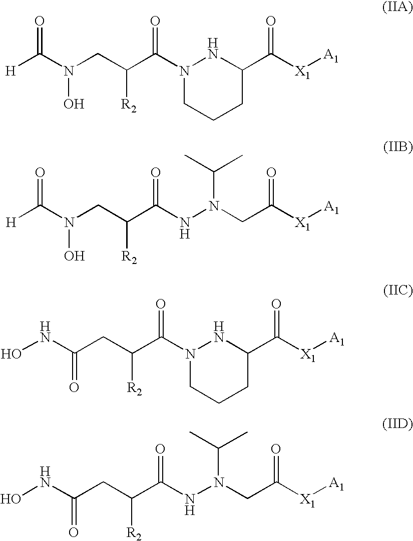
wherein R₂ represents a substituted or unsubstituted C₁-C₆ alkyl C₁-C₃ alkyl-O—C₁-C₃ alkyl, C₁-C₃ alkyl-S—C₁-C₃ alkyl, cycloalkyl(C₁-C₃ alkyl)-, aryl(C₁-C₃ alkyl)-, heterocyclyl(C₁-C₃ alkyl)-, or R¹R²N—C₁-C₃ alkyl group wherein R¹ represents hydrogen or C₁-C₃ alkyl and R² represents C₁-C₃ alkyl, or R¹ R²N-represents a cyclic amino group;
X₁ is a bond, C₁-C₃ alkylene, —NH— or —O—; and
A₁ is optionally substituted C₁-C₃ alkyl, cycloalkyl, aryl, or heterocyclic, for example methyl, ethyl phenyl, cyclopentyl, cyclohexyl, 2- or 3-furanyl, 2- or 3-thienyl, 2-, 3- or 4-pyridyl, 3-, 4- or 5-pyrazolyl, 3-, 4- or 5-oxazolyl, or 3-, 4- or 5-thiazolyl, methoxymethyl, 3,5-bis-(trifluoromethyl)phenyl, 4-trifluoromethylphenyl, 4-methoxyphenyl, 3,4-methylenedioxyphenyl, 4-fluorophenyl benzyl, 3-pyridyl, 4-pyridyl, cyclohexyl, 1,3-dimethylpyrazol-5-yl, 1-methylimidazol-5-yl, and 2-[morpholin-1-yl]pyrid-5-yl.

21. A compound as claimed in claim 20 wherein R₂ is n-propyl, n-butyl, n-pentyl, cyclopentylmethyl, cyclopentylethyl, cyclohexylmethyl or cyclohexylethyl;
X₁ is a bond, —CH₂—, —CH₂CH₂—, —CH₂CH₂CH₂—, —NH— or —O—; and
A₁ is methyl, ethyl phenyl, cyclopentyl, cyclohexyl, 2- or 3-furanyl, 2- or 3-thienyl, 2-, 3-or 4-pyridyl, 3-, 4- or 5-pyrazolyl, 3-, 4-or 5-oxazolyl, or 3-, 4-or 5-thiazolyl, methoxymethyl, 3,5-bis-(trifluoromethyl)phenyl, 4-trifluoromethylphenyl, 4-methoxyphenyl, 3,4-methylenedioxyphenyl, 4-fluorophenyl benzyl, 3-pyridyl, 4-pyridyl, cyclohexyl, 1,3-dimethylpyrazol-5-yl, 1-methylimidazol-5-yl, or 2-[morpholin-1-yl]pyrid-5-yl.

22. A method for the treatment of bacterial infections in humans and non-human mammals, which comprises administering to a subject suffering such infection an antibacterially effective dose of a compound as claimed in claim 1.

23. A method of inhibiting bacterial growth in vitro and in vivo in mammals comprising applying of compound as claimed in claim 1.

24. (canceled)

25. A method for the treatment of bacterial contamination by applying an antibacterially effective amount of a compound as claimed in claim 1 to the site of contamination.

26. A pharmaceutical or veterinary composition comprising a compound as claimed in claim 1 together with a pharmaceutically or veterinarily acceptable carrier or excipient.
